(19) United States
(12) Patent Application Publication
(10) Pub. No.: US 2005/0137669 A1
(43) Pub. Date: Jun. 23, 2005

(54) Endocardial lead for a left heart chamber

(76) Inventors: Mohan Krishnan, Shoreview, MN (US); Eric Hammill, Lauderdale, MN (US); Rebecca Aron, Minneapolis, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/731,421

(22) Filed: Dec. 9, 2003

(51) Int. Cl. A61N 1/05

(52) U.S. Cl. 607/122

(57) ABSTRACT

An endocardial lead includes a lead body extending from a proximal end to a distal end and an electrode coupled to the lead body. The lead body and the electrode each have an outer surface adapted to passively prevent formation of clots on the outer surfaces.

### FIELD

This invention relates to the field of implantable leads, and more specifically to an endocardial lead.

### BACKGROUND

Medical leads, such as cardiac leads, have a distal end having one or more electrodes and a proximal end having a terminal which is coupled to a pulse generator. Electrical therapy is delivered from the pulse generator to the heart via the electrode in order to manage cardiac rhythms. One type of therapy includes cardiac resynchronization therapy. This is typically done with the lead placed in the coronary veins. However, it can be difficult to implant leads in the coronary veins. Further, the therapy may work better with the electrical energy delivered directly to the lateral free wall of the heart. However, the concern with placing a lead in the left atrium or left ventricle is that the left atrium and ventricle pump blood directly to the brain and there is a risk of emboli and therefore stroke from an implanted device in the left chamber of the heart.

### SUMMARY

One aspect includes a lead having a lead body extending from a proximal end to a distal end and an electrode coupled to the lead body. The lead body and the electrode each have an outer surface adapted to passively prevent formation of clots on the outer surfaces.

### BRIEF DESCRIPTION OF THE DRAWINGS

FIG. 1 shows a lead in accordance with one embodiment, located in a heart.

FIG. 2 shows further details of the lead of FIG. 1.

FIG. 3 shows a partial cross-section side view of the lead of FIG. 1.

FIG. 4 shows a side view of a lead in accordance with one embodiment.

FIG. 5 shows a side view of a lead in accordance with one embodiment.

### DETAILED DESCRIPTION

The following detailed description and accompanying drawings show specific embodiments in which the present invention may be practiced. These embodiments are described in sufficient detail to enable those skilled in the art to practice the invention. Other embodiments may be utilized and structural changes may be made without departing from the scope of the present invention.

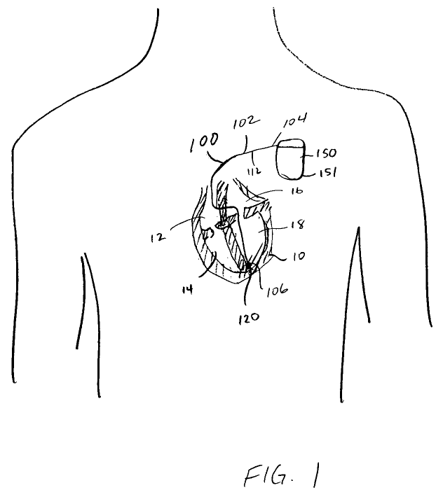

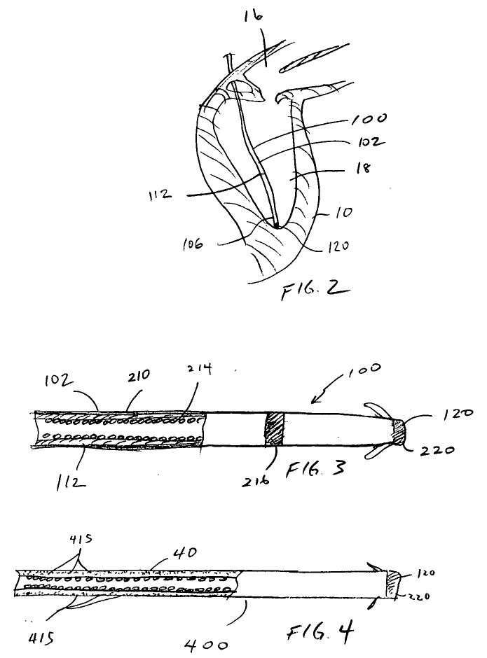

FIGS. 1 and 2 show a lead 100 according to one embodiment, implanted in a heart 10. Heart 10 generally includes a right atrium 12, a right ventricle 14, a left atrium 16, and a left ventricle 18. Lead 100 is implanted in the left ventricle 18, in this example. Lead 100 includes lead materials, lead surface coatings, and/or lead structures adapted to allow the lead to be place in the left side of the heart while mitigating risk of emboli. This design enables the placement of endocardial leads that can be implanted in the left atrium or left ventricle of the heart.

Lead 100 includes a lead body 102 extending from a proximal end 104 to a distal end 106. Proximal end 104 is coupled to a pulse generator 150. Lead 100 includes one or more conductors, such as coiled conductors or other conductors, to conduct energy from pulse generator 150 to an electrode 120, and also to receive signals from the heart. The lead further includes outer insulation 112 to insulate the conductor. The system can include a unipolar system with the case acting as an electrode or a bipolar system with a pulse between two distally located electrodes.

In one embodiment, lead 100 is adapted to deliver pacing energy to heart 10. Some examples deliver defibrillation shocks to the heart. Pulse generator 150 can be implanted in a surgically-formed pocket in a patient's chest or other desired location. Pulse generator 150 generally includes electronic components to perform signal analysis, processing, and control. Pulse generator 150 can include a power supply such as a battery, a capacitor, and other components housed in a case or can 151. The device can include microprocessors to provide processing and evaluation to determine and deliver electrical shocks and pulses of different energy levels and timing for ventricular defibrillation, cardioversion, and pacing to a heart in response to cardiac arrhythmia including fibrillation, tachycardia, and bradycardia.

One embodiment provides a lead for cardiac resynchronization therapy. The lead is adapted to be placed in the left ventricle or left atrium. The lead can be placed so that the tip electrode is located at the left lateral free wall. The lead can include tines to anchor the lead in the trebeculae in either chamber. The lead can also include a screw-in lead tip or other fixation mechanism to be placed in either chamber where there may not be trebeculae.

One procedure to place the lead is to use a transseptal approach to enter the left atrium. A transseptal needle with a dilator is inserted across the foramen ovale of the atrial septum (alternatively, the ventricular septum can be punctured). A telescoping, peel-away transseptal catheter with a needle is inserted in IVC. Then the entire assembly is pulled to the fossa ovalis using landmarks and tactile feedback. The puncture is then performed. The transseptal catheter is advance to the left side and the needle is removed. The telescoping catheter can be made shorter if necessary. The lead is then placed in catheter and placed in desired location. The catheter is withdrawn from the left side. The catheter is split, leaving the lead in place.

FIG. 3 shows a partial cross-section side view of lead 100, in accordance with one embodiment. Lead 100 includes a conductor 214, electrode 120, and a ring electrode 216. In one embodiment, lead body 102 includes an outer surface 210 adapted to passively prevent formation of clots on the outer surface. A passive coating or surface is differentiated from an active coating in that the passive surface does not elute any substance. A passive coating or surface is biologically compatible so as to prevent the body from reacting negatively to it. This allows the leads of the present system to be chronically implanted. An active coating eventually uses its entire active ingredient by eluting the ingredient. In contrast, the present passive coatings are chronic. For example, the outer surface 210 of the lead can be formed of a material that is adapted to disguise the lead body from the bloodstream in which the lead is implanted. In other words, the lead's exposed surface is designed to form a surface to mimic the cellular structure of the body (e.g. a pseudoneointimal layer) to prevent the foreign body response and subsequent clot formation.

In one embodiment, outer surface 210 of lead 100 does not include any bioactive coatings which elute from the surface to minimize clotting. The present system includes a non-eluting coating or outer surface that is inherently non-thrombogenic. This passive feature allows for a long-term implanted lead design since the lead body itself is inherently non-thrombogenic. The outer surface 210 can be the lead surface material itself or a coating on the lead, as shown in this example.

In one embodiment, lead 100 has an outer surface 210 including a phospholipid polymer. In one example, the phospholipid polymer can be an MPC polymer, such as a 10.0 wt % MPC polymer. The phospholipid polymer surface is inherently non-thrombogenic. The surface can be applied by dip-coating or spraying, for example. In one embodiment, the outer surface of the electrode 120 can also have a phospholipid polymer coating.

In one embodiment, electrode 120 includes an outer surface 220 adapted to passively prevent formation of clots on the outer surface. In one embodiment, outer surface 220 can include a textured outer surface which is adapted to trap blood cells within the textured surface to form a layer of blood cells on the electrode surface. The layer of blood cells on the electrode surface disguises the electrode within the bloodstream and prevents the blood from forming clots on the surface.

For example, one embodiment includes a coating on outer surface 220 including a plurality of microspheres, such as sintered titanium microspheres. The titanium microspheres are dimensioned to cause a region of low shear such that cells deposit and eventually form a uniform and tightly adherent biologic pseudo-neointima. One technique to apply the microspheres is to dust a coating of sieved titanium microspheres (75-100 μm dia.) on a wet surface to form a continuous layer of three to four titanium microspheres. Then they are sintered under a vacuum. In one example, ring electrode 216 can also have a textured surface, such as titanium microspheres. Some examples can include a defibrillation coil electrode that can have an outer surface textured as discussed above.

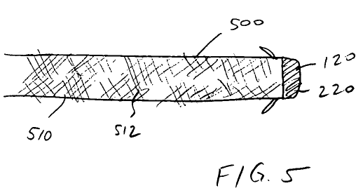

FIG. 4 shows a side view of a lead 400 in accordance with one embodiment. Lead 400 can include any of the features discussed above, and certain details will be omitted for sake of clarity. In this example, an outer surface 410 of lead 400 includes at least a portion seeded with endothelial cells 415. These endothelial cells help develop a pseudo-intimal layer on the surface of the lead when the lead is exposed to a bloodstream. Again, this layer on the lead acts so that the bloodstream does not act unfavorably to the presence of a foreign body by triggering clot formation. In one embodiment, lead outer surface 410 also includes a coating material adapted to mimic endothelial cells such that a body having the lead body implanted therein does not recognize the surface of the lead body as a foreign object. In another embodiment, stem cells can be used instead of endothelial cells.

In one embodiment, any of the leads discussed herein can have a lead body having an outer surface that includes an amino acid sequence attached to a polymer thus effecting intracellular signaling so that the sequence binds specifically to various cell surface receptors. One example is RGD which promotes the adhesion of endothelial cells and prevents fibronectin adhesion. Another example of a passive coating is polyethylene glycol tethered to the polymer surface via acrylic acid.

FIG. 5 shows a lead 500 in accordance with one embodiment. Lead 500 can include any of the features discussed above, and certain details will be omitted for sake of clarity. In this example, an outer surface 510 of lead 500 includes an outer surface including a molded polyurethane material having an irregular surface 512 which is adapted to attract blood cells to form a tight layer of cells. For example, the surface can be molded to have a textured outer surface which is adapted to trap blood cells within the textured surface to form a layer of blood cells on the electrode surface.

In some embodiments, the outer surface of the lead body material itself is textured. In other embodiments, a textured coating is provided on the lead surface. For example, one embodiment to form a textured lead includes making a negative mold (e.g. by excimer laser micromachining of negative mold). The material (such as polyurethane or silicone solution) is poured into the mold. Once the soluton has evaporated, the textured polymer can be removed. Another example is to place a mask with desired surface texture, spacing, size, etc. over the lead body. Then plasma CVD is used to deposit polymer onto the surface. The dimensions of the texture can vary. In one embodiment, the dimensions of the texture grooves are approximately 25-100 micrometers. The texture dimensions are such that the pseudo-neointamal surface formed is thin enough so nutrients can be delivered by the blood via diffusion.

In general, the lead structures discussed include materials and surfaces of materials used in the lead construction adapted such that the risk of thromboembolic complications are reduced or minimized. For example, in various embodiments, the lead body surface can be coated or textured as described herein and the electrode surface can be textured or coated as described herein.

In one example use, as discussed above, a lead according to one or more of the above examples can be implanted to provide cardiac resynchronization therapy. The lead can be inserted into the heart as discussed above. For ventricular CRT, the position of the lead can occur anywhere within the endocardial surface of the left ventricle. One method of placing the lead is to map the evoked stimuli from a right ventricular lead and locate the left ventricular lead in the are of last polarization. The lead can also be used to provide unipolar or bipolar pacing. The therapy can also include extended bipolar pacing for an electrode on the left ventricle lead to an electrode on a right ventricle lead.

In one or more embodiments, the present lead design provides a technique to passively prevent clot formation by disguising the lead so that the blood cannot detect the presence of a foreign material and trigger clot formation. This is in contrast to a bio-active approach of eluting drugs from the lead surface. Some embodiments can combine the passive techniques discussed above along with an active coating on the lead, such as heparin, for additional short-term prevention of clot formation. Moreover, although adapted for placement in a left chamber of the heart to reduce the risk of emboli, in some embodiments the leads described herein can also be used in a right heart camber, a vein, or an artery.

The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A lead comprising:
a lead body extending from a proximal end to a distal end; and
an electrode coupled to the lead body;
wherein the lead body and the electrode each have an outer surface adapted to passively prevent formation of clots on the outer surfaces.

2. The lead of claim 1, wherein the outer surface of the lead is textured so as to form a pseudo-intimal layer on the outer surface.

3. The lead of claim 1, wherein the lead body includes at least a portion seeded with endothelial cells or stem cells.

4. The lead of claim 1, wherein the lead body material includes a phospholipid polymer.

5. The lead of claim 1, wherein the outer surface of the electrode includes a textured coating or surface.

6. The lead of claim 5, wherein the electrode includes a coating including titanium microspheres.

7. The lead of claim 6, wherein the titanium microspheres are dimensioned to attract circulating blood cells so as to develop a uniform and tightly adherent biologic surface.

8. The lead of claim 1, wherein the lead body includes an amino acid sequence attached to a polymer, the amino acid sequence chosen to bind to cell receptors.

9. The lead of claim 1, wherein the outer surface of the lead does not include any active coatings which elute from the surface to minimize clotting.

10. The lead of claim 1, wherein the lead is coupled to a pulse generator and is adapted for delivering cardiac resynchronization therapy.

11. A lead comprising:
a lead body extending from a proximal end to a distal end; and
an electrode coupled to the lead body;
wherein the lead body has a textured outer surface adapted to passively prevent formation of clots on the outer surface; and
wherein the electrode includes an outer textured surface including microspheres.

12. The lead of claim 11, wherein the electrode outer surface is adapted to trap blood cells within the textured surface to form a layer of blood cells on the electrode surface.

13. The lead of claim 11, wherein the microspheres are titanium microspheres.

14. The lead of claim 11, wherein the outer surface of the lead does not include any active coatings which elute from the surface to minimize clotting.

15. The lead of claim 11, wherein the lead outer surface is inherently non-thrombogenic.

16. The lead of claim 11, wherein the lead is coupled to a pulse generator and is adapted for delivering cardiac resynchronization therapy.

17. A lead comprising:
a lead body extending from a proximal end to a distal end;
an electrode coupled to the lead body; and
means for passively preventing formation of clots on the electrode and the lead body.

18. The lead of claim 17, wherein means for passively preventing includes a microsphere outer surface coating on at least a portion of the electrode.

19. The lead of claim 17, wherein means for passively preventing includes at least a portion of the lead body having an outer surface seeded with endothelial cells or stem cells.

20. The lead of claim 17, wherein means for passively preventing includes the lead body having an outer surface including a phospholipid polymer material.

21. A method comprising:
implanting a lead in a left chamber of a heart, the lead having a non-eluting, bio-passive, non-thrombogenic outer surface;
coupling the lead to a pulse generator; and
delivering electrical pulses to the heart through the lead.

22. The method of claim 21, wherein delivering electrical pulses includes delivering CRT therapy to the heart.

23. The method of claim 22, wherein delivering CRT therapy includes sensing heart conditions through the lead.
